(19) United States
(12) Patent Application Publication
(10) Pub. No.: US 2021/0369181 A1
(43) Pub. Date: Dec. 2, 2021

(54) Method And System For Visualizing Data From Electrical Source Imaging

(71) Applicant: Persyst Development Corporation, Solana Beach, CA (US)

(72) Inventors: Nicolas Nierenberg, La Jolla, CA (US); Scott B. Wilson, Del Mar, CA (US)

(73) Assignee: Persyst Development Corporation, Solana Beach, CA (US)

(21) Appl. No.: 17/328,881

(22) Filed: May 24, 2021

Related U.S. Application Data
(60) Provisional application No. 63/031,182, filed on May 28, 2020.

(51) Int. Cl. A61B 5/369; A61B 5/055; A61B 5/291; G16H 50/50

(52) U.S. Cl. CPC A61B 5/369; G16H 50/50; A61B 5/291; A61B 5/055

(57) ABSTRACT

A method for visualizing data from electrical source imaging (ESI) is disclosed herein. The method converts the ESI into a plurality of ESI waveforms. The method generates a virtual electrode from the plurality of ESI waveforms. The method places the virtual electrode at a three-dimensional (3D) location of a representation of the patient's brain or on the surface of the scalp. The method receives a direct measurement of the virtual electrode at the 3D location.

### CROSS REFERENCE TO RELATED APPLICATION

The Present Invention claims priority to the U.S. Provisional Patent Application No. 63/031182, filed on May 28, 2020, which is hereby incorporated by reference in its entirety.

### STATEMENT REGARDING FEDERALLY SPONSORED RESEARCH OR DEVELOPMENT

Not Applicable

### BACKGROUND OF THE INVENTION

### Field of the Invention

The present invention generally relates to electrical source imaging.

### Description of the Related Art

Electrical Source Imaging (ESI) has been an important diagnostic adjunct to EEG for many years with fairly rapidly developing technology. Essentially ESI combines a model of a brain (in many cases a patient specific brain) with the scalp signals from EEG to estimate the source and intensity of the signal inside the brain.

In most cases currently ESI is used to show an image at a point in time or a moving image over a brief period of time that a three dimensional representation of the currents in the patient's brain that correspond to diagnostically relevant waveforms identified in the EEG. The most common example is for evaluating the source of an Epileptiform Spike, or an averaged group of Spikes. Since spikes are a hallmark of Epilepsy and believed to be emitted from the areas of the brain where seizures originate their location is of important diagnostic relevance. So an expert can use a model of the patient's brain, generally based on an Mill, combined with the EEG signals during the Spike to find the point of maximum activity representing the probable source of the spike.

With improvements in algorithms and computing power the models used for ESI have become much more sophisticated and detailed. This has allowed for more accurate estimation of the signals, which are now increasingly accepted as representing brain activity at particular locations.

While an image that looks something like an MM is the typical way to represent ESI it is possible to represent it in other ways. In ESI the brain is represented as a set of three dimensional voxels with each voxel receiving an estimated value at a point in time based on the scalp signals. This is technically done through a linear conversion and matrix arithmetic. The result is generally measured as a current, but can also be measured in voltage.

However these visualizations have not been widely adopted due to the choice of how the information is presented and other technical issues affecting performance etc. The current visualizations work well for a point in time or a short interval of time, but they aren't particularly suitable for the review of longer events such as seizures. This is part of the reason that the application so far has been to transient events like spikes.

Some programs have allowed the user to select a voxel or set of voxels and provided the current and/or the voltage numerically in addition to the typical colored scale representation. These programs have also let the user move to different time points with the values updating automatically to that time point.

An electroencephalogram (“EEG”) is a diagnostic tool that measures and records the electrical activity of a person's brain in order to evaluate cerebral functions. Multiple electrodes are attached to a person's head and connected to a machine by wires. The machine amplifies the signals and records the electrical activity of a person's brain. The electrical activity is produced by the summation of neural activity across a plurality of neurons. These neurons generate small electric voltage fields. The aggregate of these electric voltage fields create an electrical reading which electrodes on the person's head are able to detect and record. An EEG is a superposition of multiple simpler signals. In a normal adult, the amplitude of an EEG signal typically ranges from 1 micro-Volt to 100 micro-Volts, and the EEG signal is approximately 10 to 20 milli-Volts when measured with subdural electrodes. The monitoring of the amplitude and temporal dynamics of the electrical signals provides information about the underlying neural activity and medical conditions of the person.

An EEG is performed to: diagnose epilepsy; verify problems with loss of consciousness or dementia; verify brain activity for a person in a coma; study sleep disorders, monitor brain activity during surgery, and additional physical problems.

Multiple electrodes (typically 17-21, however there are standard positions for at least 70) are attached to a person's head during an EEG. The electrodes are referenced by the position of the electrode in relation to a lobe or area of a person's brain. The references are as follows: F=frontal; Fp=frontopolar; T=temporal; C=central; P=parietal; O=occipital; and A=auricular (ear electrode). Numerals are used to further narrow the position and “z” points relate to electrode sites in the midline of a person's head. An electrocardiogram (“EKG”) may also appear on an EEG display.

The EEG records brain waves from different amplifiers using various combinations of electrodes called montages. Montages are generally created to provide a clear picture of the spatial distribution of the EEG across the cortex. A montage is an electrical map obtained from a spatial array of recording electrodes and preferably refers to a particular combination of electrodes examined at a particular point in time.

In bipolar montages, consecutive pairs of electrodes are linked by connecting the electrode input 2 of one channel to input 1 of the subsequent channel, so that adjacent channels have one electrode in common. The bipolar chains of electrodes may be connected going from front to back (longitudinal) or from left to right (transverse). In a bipolar montage signals between two active electrode sites are compared resulting in the difference in activity recorded. Another type of montage is the referential montage or monopolar montage. In a referential montage, various electrodes are connected to input 1 of each amplifier and a reference electrode is connected to input 2 of each amplifier. In a reference montage, signals are collected at an active electrode site and compared to a common reference electrode.

Reference montages are good for determining the true amplitude and morphology of a waveform. For temporal electrodes, CZ is usually a good scalp reference.

Being able to locate the origin of electrical activity (“localization”) is critical to being able to analyze the EEG. Localization of normal or abnormal brain waves in bipolar montages is usually accomplished by identifying “phase reversal,” a deflection of the two channels within a chain pointing to opposite directions. In a referential montage, all channels may show deflections in the same direction. If the electrical activity at the active electrodes is positive when compared to the activity at the reference electrode, the deflection will be downward. Electrodes where the electrical activity is the same as at the reference electrode will not show any deflection. In general, the electrode with the largest upward deflection represents the maximum negative activity in a referential montage.

Some patterns indicate a tendency toward seizures in a person. A physician may refer to these waves as “epileptiform abnormalities” or “epilepsy waves.” These include spikes, sharp waves, and spike-and-wave discharges. Spikes and sharp waves in a specific area of the brain, such as the left temporal lobe, indicate that partial seizures might possibly come from that area. Primary generalized epilepsy, on the other hand, is suggested by spike-and-wave discharges that are widely spread over both hemispheres of the brain, especially if they begin in both hemispheres at the same time.

There are several types of brain waves: alpha waves, beta waves, delta wave, theta waves and gamma waves. Alpha waves have a frequency of 8 to 12 Hertz (“Hz”). Alpha waves are normally found when a person is relaxed or in a waking state when a person's eyes are closed but the person is mentally alert. Alpha waves cease when a person's eyes are open or the person is concentrating. Beta waves have a frequency of 13 Hz to 30 Hz. Beta waves are normally found when a person is alert, thinking, agitated, or has taken high doses of certain medicines. Delta waves have a frequency of less than 3 Hz. Delta waves are normally found only when a person is asleep (non-REM or dreamless sleep) or the person is a young child. Theta waves have a frequency of 4 Hz to 7 Hz. Theta waves are normally found only when the person is asleep (dream or REM sleep) or the person is a young child. Gamma waves have a frequency of 30 Hz to 100 Hz. Gamma waves are normally found during higher mental activity and motor functions.

The following definitions are used herein.

“Amplitude” refers to the vertical distance measured from the trough to the maximal peak (negative or positive). It expresses information about the size of the neuron population and its activation synchrony during the component generation.

The term “analogue to digital conversion” refers to when an analogue signal is converted into a digital signal which can then be stored in a computer for further processing. Analogue signals are “real world” signals (e.g., physiological signals such as electroencephalogram, electrocardiogram or electrooculogram). In order for them to be stored and manipulated by a computer, these signals must be converted into a discrete digital form the computer can understand.

“Artifacts” are electrical signals detected along the scalp by an EEG, but that originate from non-cerebral origin. There are patient related artifacts (e.g., movement, sweating, ECG, eye movements) and technical artifacts (50/60 Hz artifact, cable movements, electrode paste-related).

The term “differential amplifier” refers to the key to electrophysiological equipment. It magnifies the difference between two inputs (one amplifier per pair of electrodes).

“Duration” is the time interval from the beginning of the voltage change to its return to the baseline. It is also a measurement of the synchronous activation of neurons involved in the component generation.

“Electrode” refers to a conductor used to establish electrical contact with a nonmetallic part of a circuit. EEG electrodes are small metal discs usually made of stainless steel, tin, gold or silver covered with a silver chloride coating. They are placed on the scalp in special positions.

“Electrode gel” acts as a malleable extension of the electrode, so that the movement of the electrodes leads is less likely to produce artifacts. The gel maximizes skin contact and allows for a low-resistance recording through the skin.

The term “electrode positioning” (10/20 system) refers to the standardized placement of scalp electrodes for a classical EEG recording. The essence of this system is the distance in percentages of the 10/20 range between Nasion-Inion and fixed points. These points are marked as the Frontal pole (Fp), Central (C), Parietal (P), occipital (0), and Temporal (T). The midline electrodes are marked with a subscript z, which stands for zero. The odd numbers are used as subscript for points over the left hemisphere, and even numbers over the right

“Electroencephalogram” or “EEG” refers to the tracing of brain waves, by recording the electrical activity of the brain from the scalp, made by an electroencephalograph.

“Electroencephalograph” refers to an apparatus for detecting and recording brain waves (also called encephalograph).

“Epileptiform” refers to resembling that of epilepsy.

“Filtering” refers to a process that removes unwanted frequencies from a signal.

“Filters” are devices that alter the frequency composition of the signal.

“Montage” means the placement of the electrodes. The EEG can be monitored with either a bipolar montage or a referential one. Bipolar means that there are two electrodes per one channel, so there is a reference electrode for each channel. The referential montage means that there is a common reference electrode for all the channels.

“Morphology” refers to the shape of the waveform. The shape of a wave or an EEG pattern is determined by the frequencies that combine to make up the waveform and by their phase and voltage relationships. Wave patterns can be described as being: “Monomorphic”. Distinct EEG activity appearing to be composed of one dominant activity. “Polymorphic”. distinct EEG activity composed of multiple frequencies that combine to form a complex waveform. “Sinusoidal”. Waves resembling sine waves. Monomorphic activity usually is sinusoidal. “Transient”. An isolated wave or pattern that is distinctly different from background activity.

“Spike” refers to a transient with a pointed peak and a duration from 20 to under 70 msec.

The term “sharp wave” refers to a transient with a pointed peak and duration of 70-200 msec.

The term “neural network algorithms” refers to algorithms that identify sharp transients that have a high probability of being epileptiform abnormalities.

“Noise” refers to any unwanted signal that modifies the desired signal. It can have multiple sources.

“Periodicity” refers to the distribution of patterns or elements in time (e.g., the appearance of a particular EEG activity at more or less regular intervals). The activity may be generalized, focal or lateralized.

An EEG epoch is an amplitude of a EEG signal as a function of time and frequency.

Quantitative EEG (QEEG) was been used for some time in the analysis of EEG. The most common use is for time compressed graphical output using FFT. This type of graphical output can be interpreted by a human reader to show, for example an overview of a long period EEG in the frequency range. While a single page of EEG might display ten seconds of data, a page of QEEG might display minutes or even hours.

A voxel, as used in computer based modeling or graphic simulation, is a volume element representing a value on a regular grid in three dimensional space.

There is a need for a better way to visualize data from electrical source imaging.

### BRIEF SUMMARY OF THE INVENTION

In this invention, a new method of visualizing the data from ESI is introduced. The intention is to provide a way to visualize data over longer time periods, and in a manner that is familiar to the key users Electroencephalographers (EEGers).

One aspect of the present invention is a method for visualizing data from electrical source imaging (ESI). The method includes generating an ESI for a patient, wherein the ESI is a combination of a model of a brain with a plurality of scalp signals from an EEG that estimates a source and intensity of a signal within the patent's brain. The method also includes converting the ESI into a plurality of ESI waveforms. The method also includes generating a virtual electrode from the plurality of ESI waveforms. The method also includes placing the virtual electrode at a three-dimensional (3D) location of a representation of the patient's brain or on the surface of the scalp. The method also includes receiving a direct measurement of the virtual electrode at the 3D location.

Another aspect of the present invention is a non-transitory computer-readable medium that stores a program that causes a processor to perform functions to visual data from electrical source imaging (ESI) by executing the following steps. The first step is generating an ESI for a patient, wherein the ESI is a combination of a model of a brain with a plurality of scalp signals from an EEG that estimates a source and intensity of a signal within the patent's brain. The next step is converting the ESI into a plurality of ESI waveforms. The next step is generating a virtual electrode from the plurality of ESI waveforms. The next step is placing the virtual electrode at a three-dimensional (3D) location of a representation of the patient's brain. The next step is receiving a direct measurement of the virtual electrode at the 3D location.

Yet another aspect of the present invention is a method for visualizing data from electrical source imaging (ESI) for stereo EEG (SEEG). The method includes generating an ESI for a patient, wherein the ESI is a combination of a model of a brain with a plurality of scalp signals from an EEG that estimates a source and intensity of a signal within the patent's brain. The method also includes converting the ESI into a plurality of ESI waveforms. The method also includes generating a virtual electrode from the plurality of ESI waveforms. The method also includes placing the virtual electrode at a three-dimensional (3D) location of a representation of the patient's brain. The method also includes receiving a direct measurement of the virtual electrode at the 3D location. The method also includes generating a virtual SEEG probe based on the measurement from the virtual electrode.

Yet another aspect of the present invention is a non-transitory computer-readable medium that stores a program that causes a processor to perform functions to visual data from electrical source imaging (ESI) for stereo EEG (SEEG) by executing the following steps. The first step is generating an ESI for a patient, wherein the ESI is a combination of a model of a brain with a plurality of scalp signals from an EEG that estimates a source and intensity of a signal within the patent's brain. The next step is converting the ESI into a plurality of ESI waveforms. The next step is generating a virtual electrode from the plurality of ESI waveforms. The next step is placing the virtual electrode at a three-dimensional (3D) location of a representation of the patient's brain. The next step is receiving a direct measurement of the virtual electrode at the 3D location. The next step is generating a virtual SEEG probe based on the measurement from the virtual electrode.

Yet another aspect of the present invention is a method for visualizing data from electrical source imaging (ESI) for stereo EEG (SEEG). The method includes generating an ESI for a patient, wherein the ESI is a combination of a model of a brain with a plurality of scalp signals from an EEG that estimates a source and intensity of a signal within the patent's brain. The method also includes converting the ESI into a plurality of ESI waveforms. The method also includes generating a plurality of virtual electrodes from the plurality of ESI waveforms. The method also includes placing each of the plurality of virtual electrodes at a three-dimensional (3D) location of a representation of the patient's brain. The method also includes receiving a direct measurement from each of the plurality of virtual electrodes at the 3D location. The method also includes generating a virtual SEEG probe based on the measurement from each of the plurality of virtual electrodes.

Yet another aspect of the present invention is a non-transitory computer-readable medium that stores a program that causes a processor to perform functions to visual data from electrical source imaging (ESI) for stereo EEG (SEEG) by executing the following steps. The first step is converting an ESI into a plurality of ESI waveforms, wherein the ESI is a combination of a model of a brain with a plurality of scalp signals from an EEG that estimates a source and intensity of a signal within the patent's brain. The next step is generating a plurality of virtual electrodes from the plurality of ESI waveforms. The next step is placing each of the plurality of virtual electrodes at a three-dimensional (3D) location of a representation of the patient's brain. The next step is receiving a direct measurement from each of the plurality of virtual electrodes at the 3D location. The next step is generating a virtual SEEG probe based on the measurement from each of the plurality of virtual electrodes.

Yet another aspect of the present invention is a method for determining if a signal from a particular scalp electrode is contaminated by comparing it with its calculated value based on ESI calculations from the other electrodes.

Yet another aspect of the present invention is a method for visualizing data from electrical source imaging (ESI). The method includes converting the ESI into a plurality of ESI waveforms, wherein the ESI is a combination of a model of a brain with a plurality of scalp signals from an EEG that estimates a source and intensity of a signal within the patent's brain. The method also includes generating a plurality of virtual electrodes from the plurality of ESI waveforms. The method also includes placing each of the plurality of virtual electrodes at a three-dimensional (3D) location of a representation of the patient's brain. The method also includes receiving a direct measurement from each of the plurality of virtual electrodes at the 3D location.

Yet another aspect of the present invention is a non-transitory computer-readable medium that stores a program that causes a processor to perform functions to visual data from electrical source imaging (ESI) by executing the following steps. The first step is converting the ESI into a plurality of ESI waveforms, wherein the ESI is a combination of a model of a brain with a plurality of scalp signals from an EEG that estimates a source and intensity of a signal within the patent's brain. The next step is generating a plurality of virtual electrodes from the plurality of ESI waveforms, The next step is placing each of the plurality of virtual electrodes at a three-dimensional (3D) location of a representation of the patient's brain. The next step is receiving a direct measurement from each of the plurality of virtual electrodes at the 3D location.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is a is a block diagram of a method for visualizing data from ESI.

FIG. 2 is an image of a quantitative EEG.

FIG. 3 is an illustration of a system for calculating a quantitative EEG with a patient having an open electrode.

FIG. 3A is an illustration of an isolated view of a patient with an open electrode.

FIG. 4 is a map for electrode placement for an EEG.

FIG. 5 is a detailed map for electrode placement for an EEG.

FIG. 6 is an illustration of a CZ reference montage.

FIG. 7 is an illustration of an EEG recording containing a seizure, a muscle artifact and an eye movement artifact.

FIG. 8 is an illustration of the EEG recording of FIG. 7 with the muscle artifact removed.

FIG. 9 is an illustration of the EEG recording of FIG. 8 with the eye movement artifact removed.

FIG. 10 is a flow chart for a method for visualizing data from ESI.

FIG. 11 is a flow chart for a method for visualizing data from ESI for SEEG.

FIG. 12 is an illustration of a system for calculating a quantitative EEG.

FIG. 13 is a block diagram of a system for calculating a quantitative EEG.

FIG. 14 is a flow chart for a method for visualizing data from ESI for SEEG.

FIG. 15 is a flow chart for a method for visualizing data from ESI.

### DETAILED DESCRIPTION OF THE INVENTION

The present invention is a new method of visualizing the data from ESI. The invention provides a way to visualize data over longer time periods, and in a manner that is familiar to the key users electroencephalographers. (EEGers).

The primary diagnostic tool for epilepsy is the EEG. EEGers are trained over long periods of time to recognize the fundamental waveforms in an EEG recording and differentiate artifact from cerebral signal, and diagnostically relevant cerebral signals from the background. They can do this reliably and at high speed after years of medical training. In this invention we convert the results of ESI into waveforms so that the user can directly utilize these skills in interpretation. This will be particularly valuable in reviewing longer term events such as seizures.

At the core of the invention is the concept of a virtual electrode. A virtual electrode could be placed by a user anywhere in the brain with the ESI results measured in micro-volts (mV) presented as a time series in parallel to the actual scalp EEG. Micro-volts is chosen because EEG is represented in micro-volts and therefore the time series will look precisely like what an EEGer has been trained to view. But in this case instead of having to interpret the meaning of a set of scalp electrodes placed very far away from the relevant portion of the brain, the EERer will see an estimated direct measurement at a point of diagnostic interest. Users also can specify a multiplicity of virtual electrodes allowing for direct review at different points in the brain in parallel.

In epilepsy diagnostics, a patient's EEG is initially recorded non-invasively using scalp electrodes. Depending on the treatment path, patients eventually may be implanted with electrodes using a technique called Stereo EEG, or SEEG. In this technique a burr hole is drilled in the patients skull and a sensor “probe” is placed deeply into the patient's brain. On the probe, electrodes are spaced at known distances, typically from 2-10 mm. Several of these probes are generally implanted at once and the EEG is recorded for the patient over an extended time period. Generally the hope is that seizures will be captured and using these invasive electrodes, the seizure onset zone is more accurately identified.

An alternative embodiment is a virtual SEEG. In this embodiment, the user is provided with a way to simulate the implantation of one or more SEEG probes with the virtual electrode positions determined by the characteristics and placement of the probe. These virtual electrodes are added to the EEG display for the patient, thus simulating what would be seen in the case of an actual implantation. Depending on the scalp electrode count, there is less resolution than with the actual implanted electrodes, but the EEGer is able to use this to make predictions about what would be seen by any given choice of actual SEEG probe. Frequently the exact choice of position and quantity of probes is a difficult one to make. The desire is to implant the minimum necessary to locate the likely seizure onset zone.

The virtual electrode is a 3D coordinate location inside the patient's brain along with a circumference representing the area to be sampled. The idea is to have sets of these virtual electrodes constructed in arrays that match the types of implants used in intracranial EEG monitoring. These are termed grids and strips for subdural recording, and depth arrays used in stereo EEG recording. By placing these into an image of the patient's brain, a set of virtual electrode locations are established. The user could “implant” one or more virtual sets resulting in an array of electrode locations. This array would be displayed on an EEG page that looks like the page that is produced by actual invasive recordings.

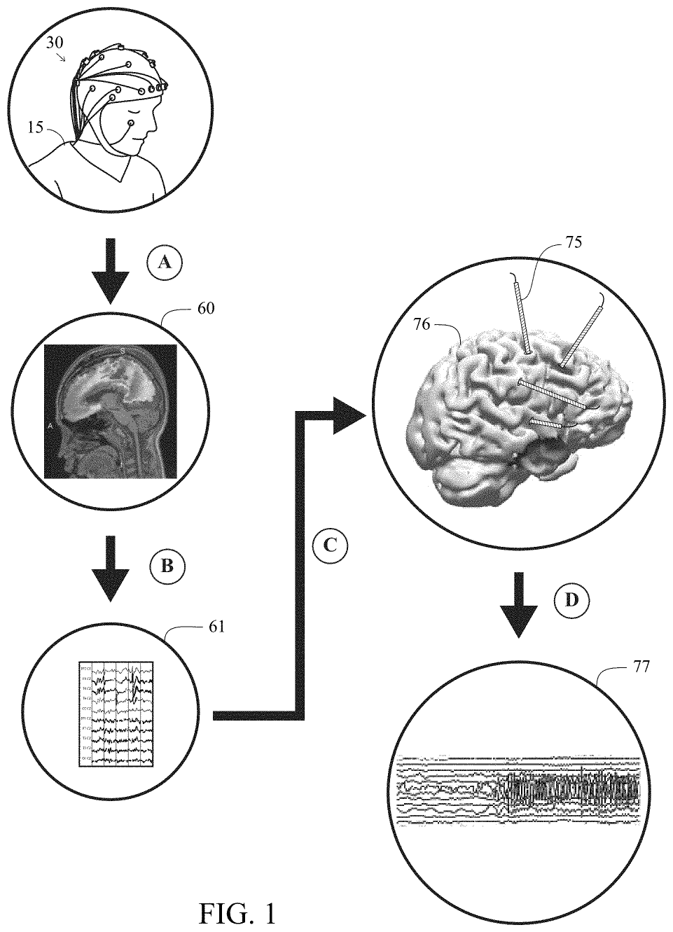

FIG. 1 illustrates the method for visualizing data from ESI. An ESI 60 is generated for a patient 15 in step A. The ESI 60 is preferably a combination of a model of a brain with scalp signals from an EEG that estimates a source and intensity of a signal within the patient's brain. The ESI 60 is converted into ESI waveforms 61 in step B. In step C, a virtual electrode 75 is generated from the ESI waveforms 61, and placed at a 3D location of a representation of the patient's brain 76 or on the surface of the scalp. In step D, a direct measurement 77 of the virtual electrode 75 at the 3D location is received.

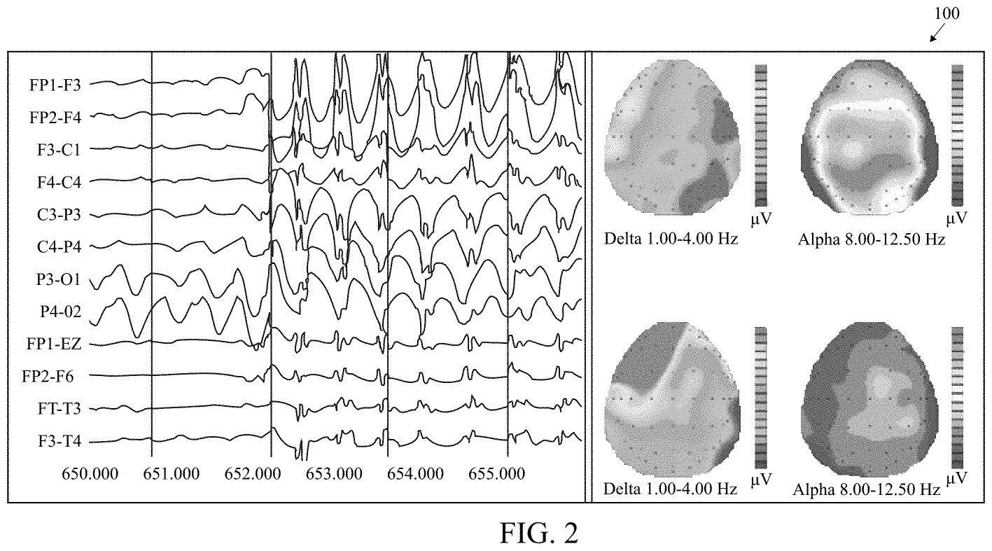

In addition to being able to display the simulated EEG at a virtual electrode 75 position it is possible to provide other features typically present in EEG systems such as the ability to re-montage, and to perform analytics such as Quantitative EEG (qEEG) 100, as shown in FIG. 2, an example of a QEEG 100.

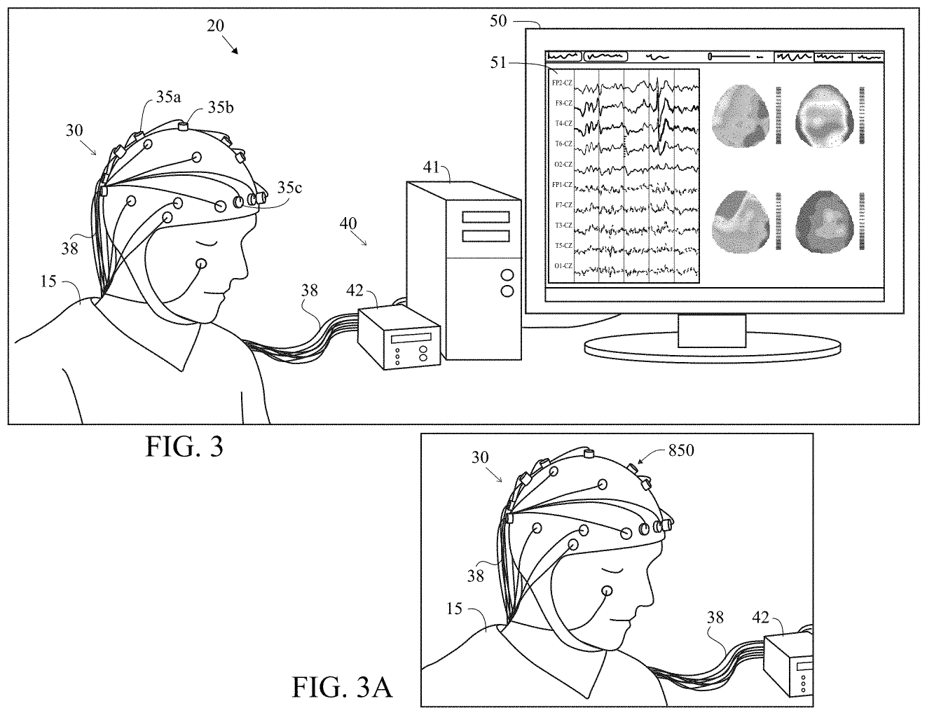

In a system 20 for calculating a quantitative EEG, as shown in FIG. 3, a patient 15 wears an electrode cap 30, consisting of a plurality of electrodes 35a-35c, attached to the patient's head with wires 38 from the electrodes 35 connected to an EEG machine component 40 which consists of an amplifier 42 for amplifying the signal to a computer 41 with a processor, which is used to analyze the signals from the electrodes 35 and generate an EEG recording 51 and a qEEG, which can be viewed on a display 50. As shown in FIG. 3A, an electrode 850 is open, unattached, and over an impedance threshold value. Thus, if the signal from that electrode 850 is included in a qEEG, the qEEG value would be inaccurate. A more thorough description of an electrode utilized with the present invention is detailed in Wilson et al., U.S. Pat. No. 8,112,141 for a Method And Device For Quick Press On EEG Electrode, which is hereby incorporated by reference in its entirety. The EEG is optimized for automated artifact filtering. The EEG recordings are then processed using neural network algorithms to generate a processed EEG recording which is used to generate a qEEG.

An additional description of analyzing EEG recordings is set forth in Wilson et al., U.S. patent application Ser. No. 13/620855, filed on Sep. 15, 2012, for a Method And System For Analyzing An EEG Recording, which is hereby incorporated by reference in its entirety.

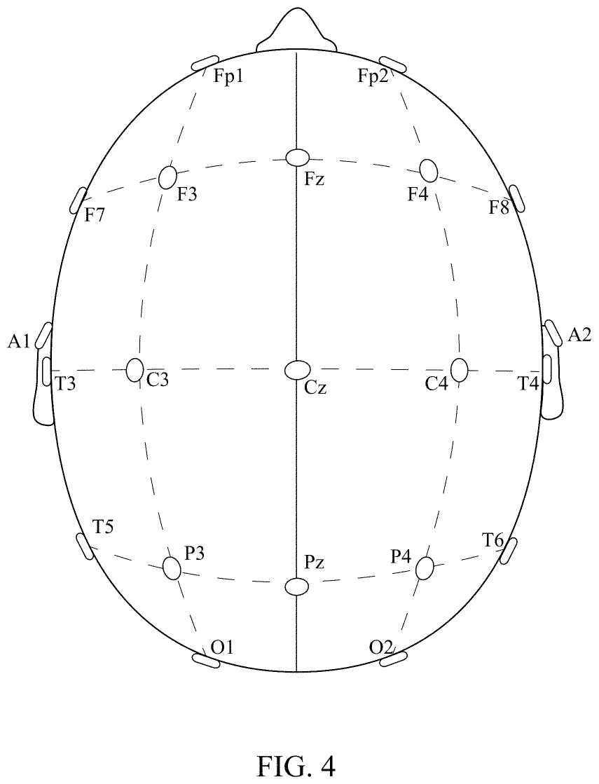

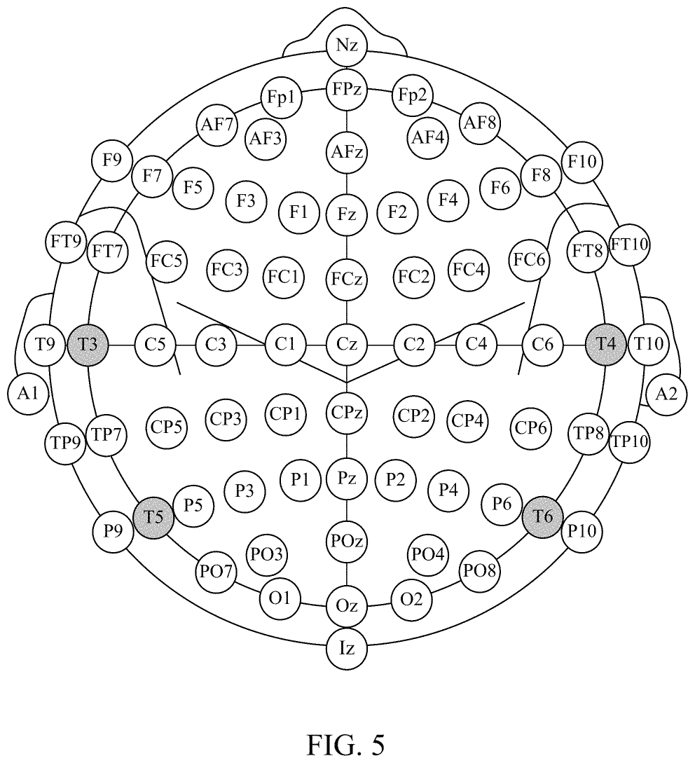

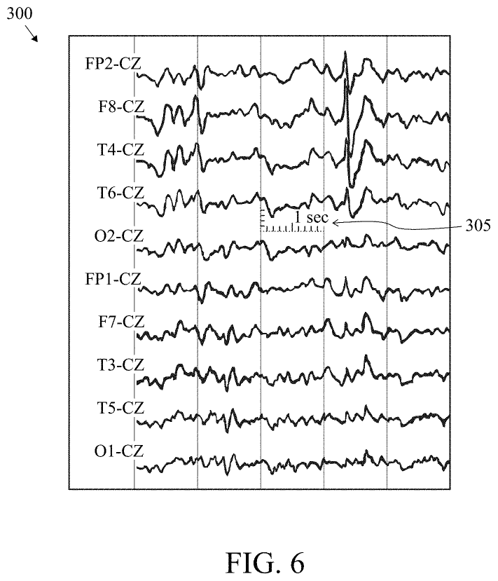

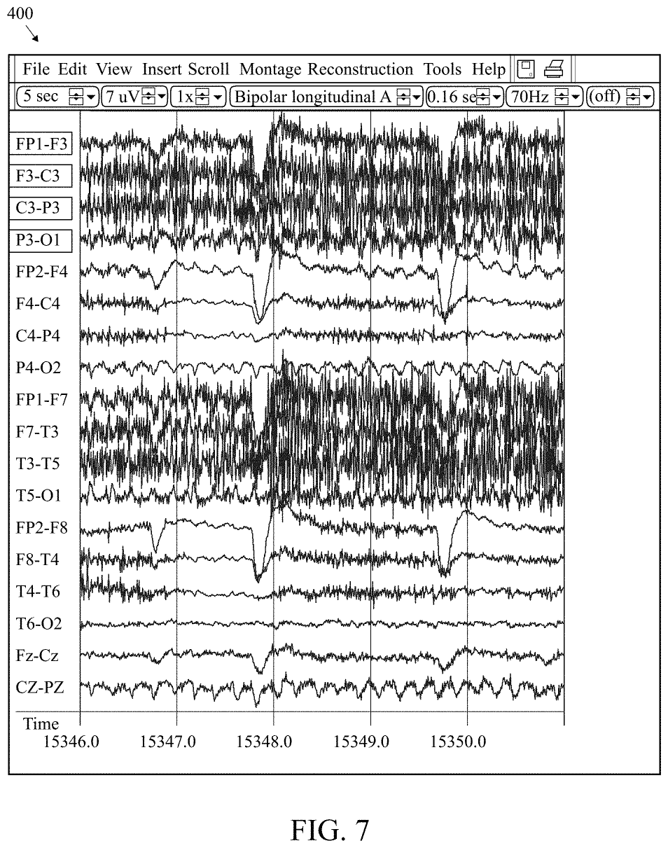

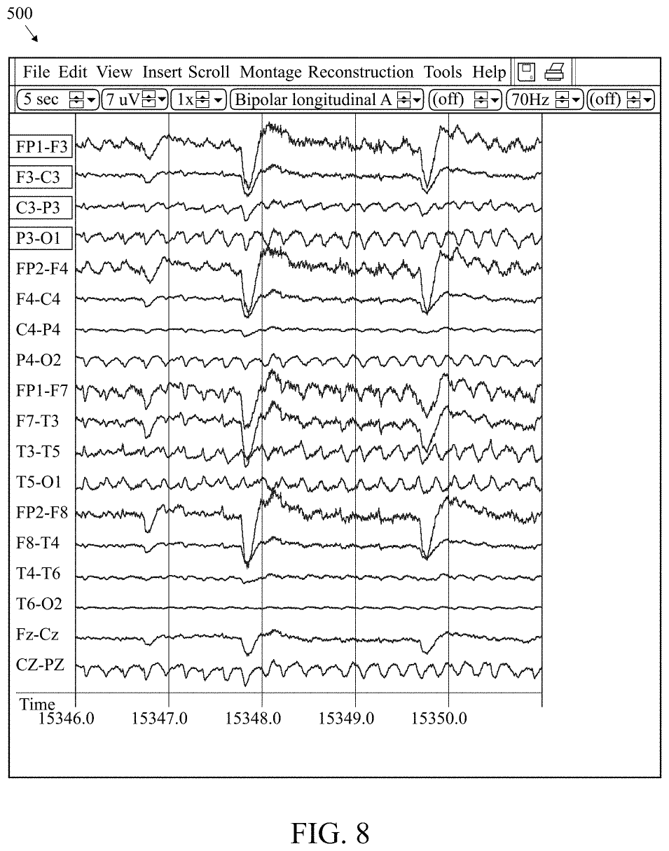

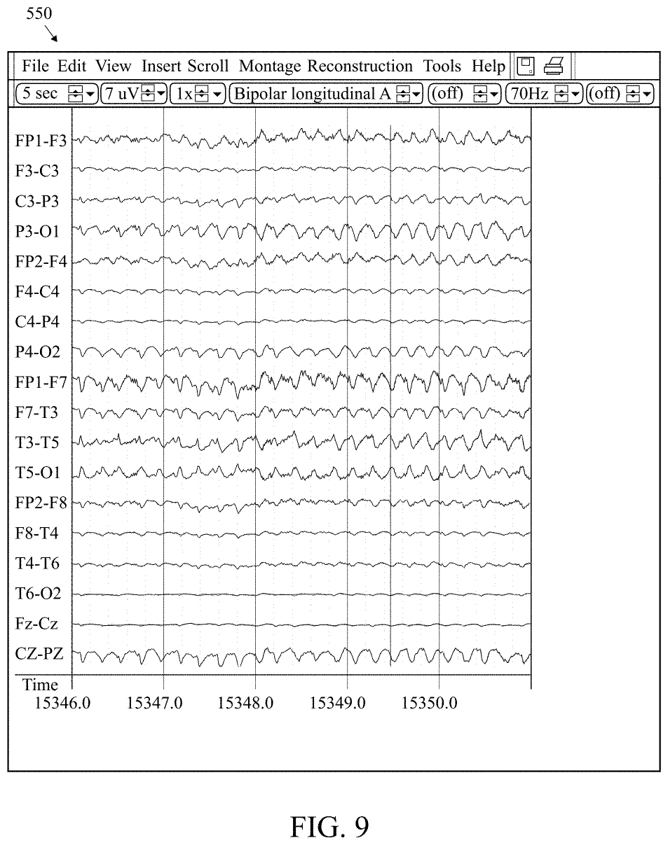

A patient has a plurality of electrodes attached to the patient's head with wires from the electrodes connected to an amplifier for amplifying the signal to a processor, which is used to analyze the signals from the electrodes and create an EEG recording. The brain produces different signals at different points on a patient's head. Multiple electrodes are positioned on a patient's head as shown in FIGS. 4 and 5. The CZ site is in the center. For example, Fp1 on FIG. 5 is represented in channel FP1-F3 on FIG. 7. The number of electrodes determines the number of channels for an EEG. A greater number of channels produce a more detailed representation of a patient's brain activity. If an electrode is open, then the recording for the channel is inaccurate thereby generating false readings. Preferably, each amplifier 42 of an EEG machine component 40 corresponds to two electrodes 35 attached to a head of the patient 15. The output from an EEG machine component 40 is the difference in electrical activity detected by the two electrodes. The placement of each electrode is critical for an EEG report since the closer the electrode pairs are to each other, the less difference in the brainwaves that are recorded by the EEG machine component 40. A more thorough description of an electrode utilized with the present invention is detailed in Wilson et al., U.S. Pat. No. 8,112,141 for a Method And Device For Quick Press On EEG Electrode, which is hereby incorporated by reference in its entirety.

The EEG is optimized for automated artifact filtering. The EEG recordings are then processed using neural network algorithms to generate a processed EEG recording, which is analyzed for display. During acquisition of the EEG recording, a processing engine performs continuous analysis of the EEG waveforms and determines the presence of most types of electrode artifact on a channel-by-channel basis. Much like a human reader, the processing engine detects artifacts by analyzing multiple features of the EEG traces. The preferred artifact detection is independent of impedance checking. During acquisition the processing monitors the incoming channels looking for electrode artifacts. When artifacts are detected they are automatically removed from the seizure detection process and optionally removed from the trending display. This results in much a much higher level of seizure detection accuracy and easier to read trends than in previous generation products.

Algorithms for removing artifact from EEG typically use Blind Source Separation (BSS) algorithms like CCA (canonical correlation analysis) and ICA (Independent Component Analysis) to transform the signals from a set of channels into a set of component waves or “sources.”

In one example an algorithm called BSS-CCA is used to remove the effects of muscle activity from the EEG. Using the algorithm on the recorded montage will frequently not produce optimal results. In this case it is generally optimal to use a montage where the reference electrode is one of the vertex electrodes such as CZ in the international 10-20 standard. In this algorithm the recorded montage would first be transformed into a CZ reference montage prior to artifact removal. In the event that the signal at CZ indicates that it is not the best choice then the algorithm would go down a list of possible reference electrodes in order to find one that is suitable.

It is possible to perform BSS-CCA directly on the user-selected montage. However, this has two issues. First this requires doing an expensive artifact removal process on each montage selected for viewing by the user. Second the artifact removal will vary from one montage to another, and will only be optimal when a user selects a referential montage using the optimal reference. Since a montage that is required for reviewing an EEG is frequently not the same as the one that is optimal for removing artifact this is not a good solution.

Various trends for an EEG recording are generated by a processing engine. A seizure probability trend, a rhythmicity spectrogram, left hemisphere trend, a rhythmicity spectrogram, right hemisphere trend, a FFT spectrogram left hemisphere trend, a FFT spectrogram right hemisphere trend, an asymmetry relative spectrogram trend, an asymmetry absolute index trend, an aEEG trend, and a suppression ration, left hemisphere and right hemisphere trend.

Rhythmicity spectrograms allow one to see the evolution of seizures in a single image. The rhythmicity spectrogram measures the amount of rhythmicity which is present at each frequency in an EEG record.

The seizure probability trend shows a calculated probability of seizure activity over time. The seizure probability trend shows the duration of detected seizures, and also suggests areas of the record that may fall below the seizure detection cutoff, but are still of interest for review. The seizure probability trend when displayed along with other trends, provides a comprehensive view of quantitative changes in an EEG.

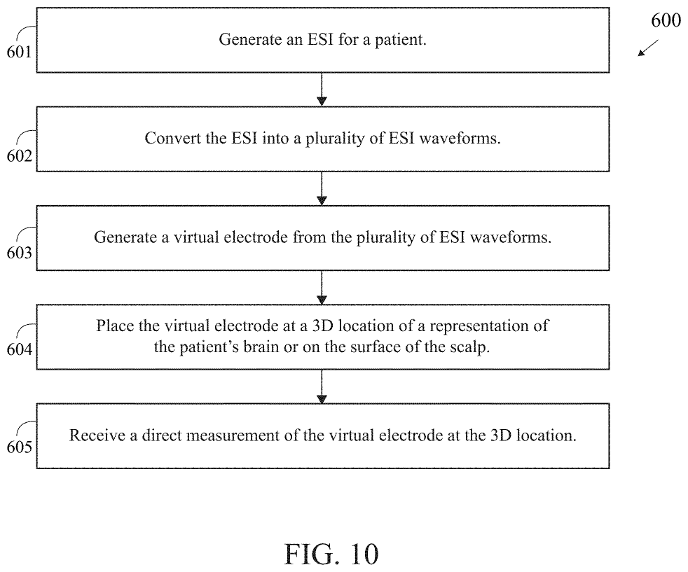

A method for visualizing data from ESI is generally designated 600 in FIG. 10. At block 601, an ESI for a patient is generated. The ESI is a combination of a model of a brain with a plurality of scalp signals from an EEG that estimates a source and intensity of a signal within the patient's brain. At block 602, the ESI is converted into a plurality of ESI waveforms. At block 603, a virtual electrode is generated from the plurality of ESI waveforms. At block 604, the virtual electrode is placed at a 3D location of a representation of the patient's brain or on the surface of the scalp. At block 605, a direct measurement of the virtual electrode at the 3D location is received.

The ESI of the method 600 preferably comprises MRI imaging. The ESI model of the patient's brain is preferably created prior to the acquisition of an EEG.

The method 600 further comprises improving seizure and spike detection performance for an EEG, and determining if there are more than one cluster of spikes for the patient.

EEG signals are generated from an EEG machine comprising a plurality of electrodes, an amplifier and processor. The EEG signals are processed continuously for artifact reduction to generate a processed EEG recording. A quantitative EEG is calculated from the processed EEG recording. Preferably, Fast Fourier Transform signal processing is used to compute the quantitative EEG. The reduced artifact types are selected from the group comprising an eye blink artifact, a muscle artifact, a tongue movement artifact, a chewing artifact, and a heartbeat artifact.

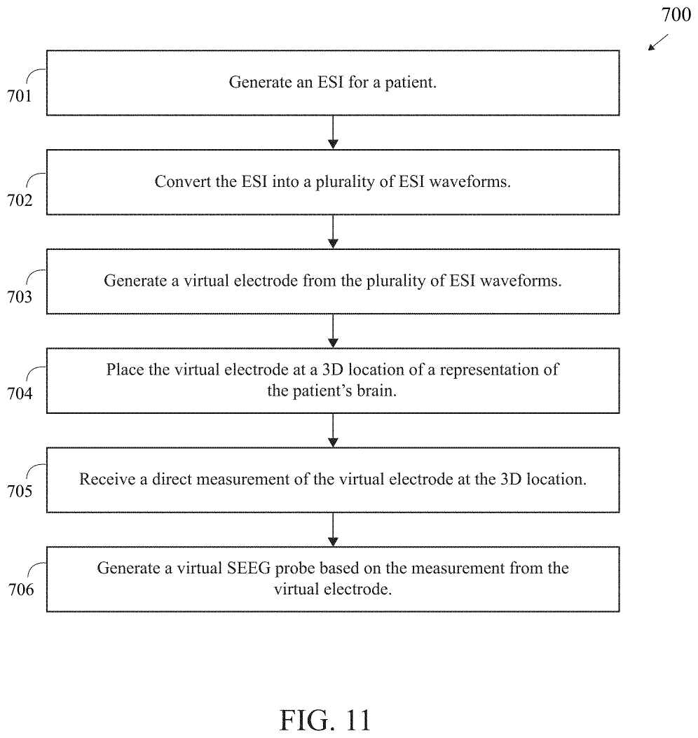

As shown in FIG. 11, a method for visualizing data from ESI for stereo EEG (SEEG) is generally designated 700. At block 701, an ESI for a patient is generated, wherein the ESI is a combination of a model of a brain with a plurality of scalp signals from an EEG that estimates a source and intensity of a signal within the patient's brain. At block 702, the ESI is converted into a plurality of ESI waveforms. A virtual electrode is generated from the plurality of ESI waveforms at block 703. At block 704, the virtual electrode is placed at a 3D location of a representation of the patient's brain. At block 705, a direct measurement of the virtual electrode at the 3D location is received. At block 706, a virtual SEEG probe based on the measurement from the virtual electrode is generated.

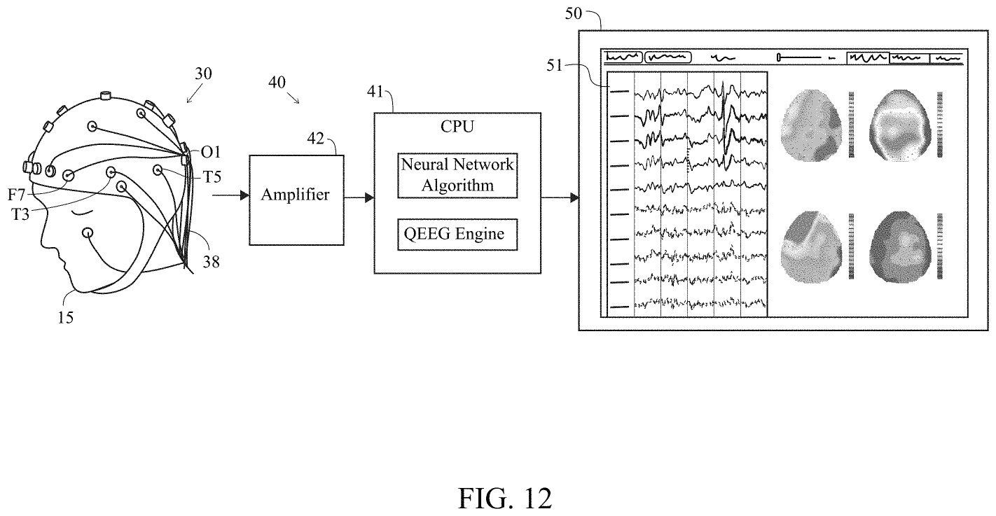

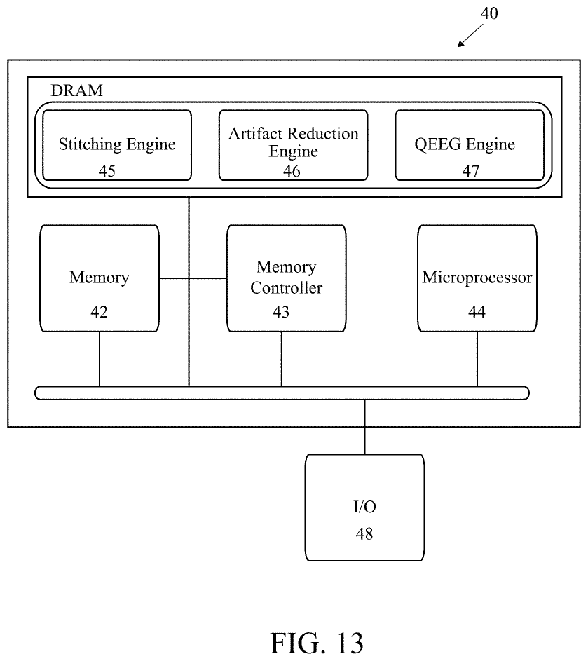

In a system for calculating a quantitative EEG, as shown in FIG. 12, a patient 15 wears an electrode cap 30, consisting of a plurality of electrodes 35a-35c, attached to the patient's head with wires 38 from the electrodes 35 connected to an EEG machine component 40 which consists of an amplifier 42 for amplifying the signal to a computer 41 with a processor, which is used to analyze the signals from the electrodes 35 and generate an EEG recording and a qEEG 51, which can be viewed on a display 50. The CPU 41 includes a software program for a neural network algorithm and a software program for a qEEG engine. As shown in FIG. 13, an artifact reduction engine 46, a qEEG engine 47, a microprocessor 44, a memory 42, a memory controller 43 and an I/O 48 are components of the EEEG machine 40. A more thorough description of an electrode utilized with the present invention is detailed in Wilson et al., U.S. Pat. No. 8,112,141 for a Method And Device For Quick Press On EEG Electrode, which is hereby incorporated by reference in its entirety. The EEG is optimized for automated artifact filtering. The EEG recordings are then processed using neural network algorithms to generate a processed EEG recording which is analyzed for display.

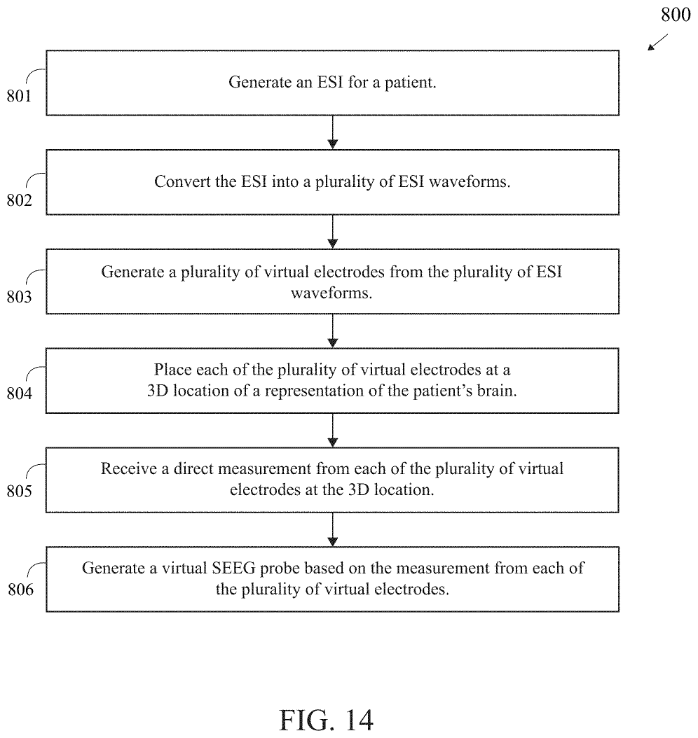

A method for visualizing data from ESI for SEEG is generally designated 800 in FIG. 14. At block 801, an ESI for a patient is generated. At block 802, the ESI is converted into a plurality of ESI waveforms. A plurality of virtual electrodes is generated from the plurality of ESI waveforms at block 803. At block 804, each of the plurality of virtual electrodes is placed at a 3D location of a representation of the patient's brain. At block 805, a direct measurement from each of the virtual electrodes at the 3D location is received. At block 806, a virtual SEEG probe based on the measurement from each of the virtual electrodes is generated.

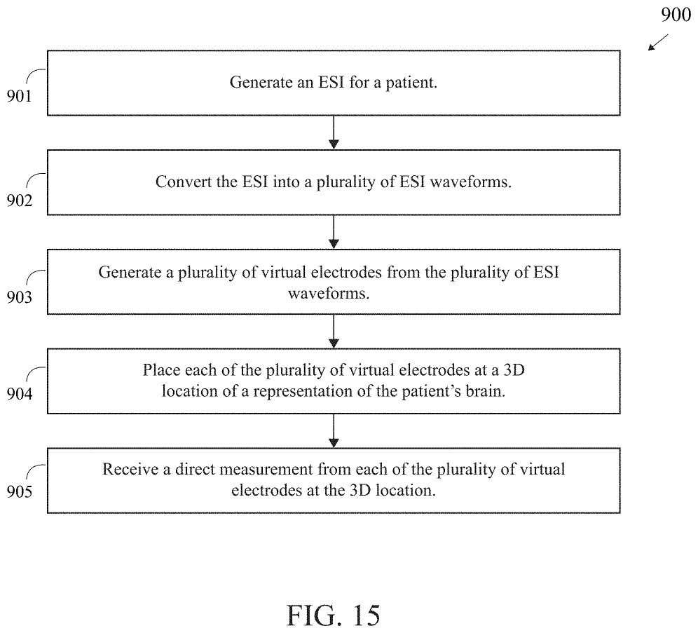

A method for visualizing data from ESI is generally designated 900 in FIG. 15. At block 901, an ESI for a patient is generated. At block 902, the ESI is converted into a plurality of ESI waveforms. A plurality of virtual electrodes is generated from the plurality of ESI waveforms at block 903. At block 904, each of the plurality of virtual electrodes is placed at a 3D location of a representation of the patient's brain. At block 905, a direct measurement from each of the virtual electrodes at the 3D location is received.

A more thorough description of EEG analysis utilized with the present invention is detailed in Wilson et al., U.S. patent application Ser. No. 13/620855, filed on Sep. 15, 2012, for a Method And System For Analyzing An EEG Recording, which is hereby incorporated by reference in its entirety. A more thorough description of a user interface utilized with the present invention is detailed in Wilson et al., U.S. Pat. No. 9,055,927, for a User Interface For Artifact Removal In An EEG, which is hereby incorporated by reference in its entirety. An additional description of analyzing EEG recordings is set forth in Wilson et al., U.S. patent application Ser. No. 13/684556, filed on Nov. 25, 2012, for a Method And System For Detecting And Removing EEG Artifacts, which is hereby incorporated by reference in its entirety. A more thorough description of displaying an EEG utilized with the present invention is detailed in Nierenberg et al., U.S. Pat. No. 8,666,484, for a Method And System For Displaying EEG Recordings, which is hereby incorporated by reference in its entirety. A more thorough description of displaying EEG recordings utilized with the present invention is detailed in Wilson et al., U.S. Pat. No. 9,232,922, for a User Interface For Artifact Removal In An EEG, which is hereby incorporated by reference in its entirety. An additional description of qEEG is set forth in Nierenberg et al., U.S. patent application Ser. No. 13/830742, filed on Mar. 14, 2013, for a Method And System To Calculate qEEG, which is hereby incorporated by reference in its entirety. An additional description of using neural networks with the present invention is set forth in Wilson, U.S. patent application Ser. No. 14/078497, filed on Nov. 12, 2013, for a Method And System Training A Neural Network, which is hereby incorporated by reference in its entirety. An additional description of using neural networks with the present invention is set forth in Nierenberg et al., U.S. patent application Ser. No. 14/222655, filed on Jan. 20, 2014, for a System And Method For Generating A Probability Value For An Event, which is hereby incorporated by reference in its entirety. Wilson et al., U.S. patent application Ser. No. 16/294917, filed on Mar. 7, 2019, for a Method And System For Utilizing Empirical Null Hypothesis For a Biological Time Series, which is hereby incorporated by reference in its entirety. Wilson et al., U.S. patent application Ser. No. 16/288731, filed on Feb. 28, 2019, for a Graphically Displaying Evoked Potentials, which is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention the following:

1. A method for visualizing data from electrical source imaging (ESI), the method comprising:
converting an ESI for a patient into a plurality of ESI waveforms, wherein the ESI is a combination of a model of a brain with a plurality of scalp signals from an EEG that estimates a source and intensity of a signal within the patent's brain;
generating a virtual electrode from the plurality of ESI waveforms;
placing the virtual electrode at a three-dimensional (3D) location of a representation of the patient's brain or on the surface of the scalp; and
receiving a direct measurement of the virtual electrode at the 3D location.

2. The method according to claim 1 wherein the ESI comprises MRI imaging.

3. The method according to claim 1 wherein the ESI model of the patient's brain is created prior to the acquisition of an EEG.

4. The method according to claim 1 further comprising improving seizure and spike detection performance for an EEG.

5. The method according to claim 1 further comprising determining if there are more than one cluster of spikes for the patient.

6. A non-transitory computer-readable medium that stores a program that causes a processor to perform functions to visual data from electrical source imaging (ESI) by executing the following steps:
converting an ESI for a patient into a plurality of ESI waveforms, wherein the ESI is a combination of a model of a brain with a plurality of scalp signals from an EEG that estimates a source and intensity of a signal within the patent's brain;
generating a virtual electrode from the plurality of ESI waveforms;
placing the virtual electrode at a three-dimensional (3D) location of a representation of the patient's brain; and
receiving a direct measurement of the virtual electrode at the 3D location.

7. The non-transitory computer readable medium according to claim 6 wherein the ESI comprises MRI imaging.

8. The non-transitory computer readable medium according to claim 6 wherein the ESI model of the patient's brain is created prior to the generating an EEG.

9. The non-transitory computer readable medium according to claim 6 further comprising improving seizure and spike detection performance for an EEG.

10. The non-transitory computer readable medium according to claim 6 further comprising determining if there are more than one cluster of spikes for the patient.

11. A method for visualizing data from electrical source imaging (ESI) for stereo EEG (SEEG), the method comprising:
converting a ESI for a patient into a plurality of ESI waveforms, wherein the ESI is a combination of a model of a brain with a plurality of scalp signals from an EEG that estimates a source and intensity of a signal within the patent's brain;
generating a virtual electrode from the plurality of ESI waveforms;
placing the virtual electrode at a three-dimensional (3D) location of a representation of the patient's brain;
receiving a direct measurement of the virtual electrode at the 3D location;
generating a virtual SEEG probe based on the measurement from the virtual electrode.
